(19) United States
(12) Patent Application Publication
(10) Pub. No.: US 2025/0119717 A1
(43) Pub. Date: Apr. 10, 2025

(54) OBJECT ASSOCIATION USING PROXIMITY SENSING

(71) Applicant: EATON INTELLIGENT POWER LIMIT ED, Dublin (IE)

(72) Inventors: Padhraig Ryan, Kilkenny (IE); Daniel Zucchetto, Dublin (IE)

(21) Appl. No.: 18/695,483

(22) PCT Filed: Oct. 18, 2021
(86) PCT No.: PCT/EP2021/078851

§ 371 Date: Mar. 26, 2024

(51) Int. Cl. H04W 4/80; G16H 40/20; H04W 4/02

(52) U.S. Cl. CPC H04W 4/80; G16H 40/20; H04W 4/023

(57) ABSTRACT

Some embodiments relate to a method of associating objects using proximity sensing is described. Each of the objects to be associated is adapted to emit signals using a short-range communications technology. Each of the objects continually emits a signal comprising an identifier of that object. A plurality of detecting devices detect signals emitted from the objects. Distances between the emitting object and the detecting device are determined for each of the detected signals. Clusters of the objects are determined from the determined distances. An attempt is then made to associate each cluster with an event. For each cluster associated with the event, the duration and composition of the cluster are determined. Devices suitable for acting as detecting devices and objects are described, as is a computer system for carrying out the method.

### CROSS-REFERENCE TO RELATED APPLICATIONS

This application is a national phase filing under 35 C.F.R. § 371 of and claims priority to PCT Patent Application No. PCT/EP2021/078851, filed on Oct. 18, 2021.

### TECHNICAL FIELD

The disclosure relates to methods and apparatus for object association using proximity sensing. It is particularly relevant to proximity sensing using wireless communication devices.

### BACKGROUND

It is important in a number of real-world applications to detect the distance between two individuals or objects. For example, in robotics it may be important to detect distance between objects in order to navigate a path through obstacles. Such a determination may be used in proximity sensing using electronic devices, which has become increasingly important in a range of contexts, such as social distancing and contact tracing. Typical technologies include short-range wireless technologies such as Bluetooth Low Energy (Bluetooth LE). Other personal area network technologies can be used, but Bluetooth LE is particularly effective because of its low power consumption in maintaining a broadly consistent communication range.

A particular problem in this connection is the determination of whether a particular electronic device is associated with a particular event in an environment. Typically, this will simply be determined by proximity of the electronic device to a location associated with the event. Where the situation is complex, however, this may not be an effective approach to take, meaning that proximity sensing may not be used to address certain classes of real-world problem. It would be desirable to develop approaches to object association that will allow effective association of electronic devices with events, particularly where the electronic devices are part of a complex assemblage of electronic devices.

### SUMMARY OF DISCLOSURE

In a first aspect, the disclosure provides a method of associating objects using proximity sensing, where each of the objects to be associated is adapted to emit signals using a short-range communications technology, the method comprising: each of the objects continually emitting a signal comprising an identifier of that object; a plurality of detecting devices detecting signals emitted from the objects; from detected signals from one or more of the detecting devices, determining distances between the emitting object and the detecting device for each of the detected signals; from the determined distances, determining clusters of the objects; attempting to associate each cluster with an event; and for each cluster associated with the event, determining the duration and composition of the cluster.

Here, the plurality of detecting devices may comprise one or more of the objects, and it may comprise a detecting device associated with a location of the event. The event may be one of a plurality of events and attempting to associate each cluster with an event may comprise attempting to associate each cluster with any of the plurality of events.

Using this approach, individuals—represented by the “emitting objects”, which may for example be implemented as mobile telephones—can be matched with events—such as the presence of a patient in a hospital bed at a particular time—in an effective way, even in a complex environment such as a hospital ward round where multiple clinicians or other medical personnel may be evaluating a number of patients in turn. In this way, clinician time (and cost) may be associated with a particular patient effectively.

Here, the short-range communications technology may be a radio technology. If so, determining distances between the emitting object and the detecting device may comprise determining distances from received signal strength indicators of the detected signals. The radio technology may be Bluetooth LE (Bluetooth Low Energy).

Using this approach, determining clusters of the objects may comprise the following: determination of whether two objects are within a predetermined proximity threshold, assigning those two objects are a cluster, determining whether any further objects are within the predetermined proximity threshold of objects in the cluster and adding such further objects into the cluster, and performing the step of determining whether any further objects are within the predetermined proximity threshold of the objects in the cluster until an end condition is reached. An obvious end condition is lack of any further objects within the predetermined proximity threshold of any device in the cluster. However, another possible end condition is that a predetermined cumulative distance threshold defining maximum cluster size is reached.

Allocation of the cluster to one of the plurality of events may comprise allocating the cluster to the event closest to a mean position of the objects in the cluster. Alternatively, it may comprise allocating the cluster to the event closest to a determined key object of the plurality of objects. In some cases, allocation of the cluster to one of the plurality of events may comprise providing confidence scores for the allocation of the cluster to one of the plurality of the events and others of the plurality of events.

As previously discussed, the method may be carried out in respect of a medical care environment, in which case the objects may be physically associated with medical professionals, and the events may be each associated with a patient in the medical care environment.

In a second aspect, the disclosure provides a computing device having a processor, a memory, and a short-range communication apparatus, wherein the processor of the computing device is programmed to: continually emit a signal comprising an identifier for the computing device using the short-range communication apparatus; detect signals comprising identifiers of other computing devices using the short-range communication apparatus; and store in the memory a record of said detected signals for determination of whether the computing device and other computing devices have formed a cluster.

The computing device may be further adapted to upload the record of detected signals to a cluster determining resource when an upload condition is met. This upload condition may be one of a data volume condition or a time condition.

The short-range communication apparatus may be a radio apparatus, and if so it may use a Bluetooth Low Energy wireless communication technology.

In a third aspect, the disclosure provides a computer system comprising a processing system, a memory and a communications system, wherein the processing system is programmed to: receive detection records from a plurality of detecting devices, wherein each detection record comprises time and signal information for detected signals comprising object identifiers of emitting objects, wherein each detection record is also associated with an object or location identifier of a detecting device; determine from the detection records distances between the emitting object and the detecting device for each of the detected signals; from the determined distances, determine clusters of the objects; attempt to associate each cluster with an event; and for each cluster associated with the event, determine the duration and composition of the cluster.

Here, the short-range communications technology may be a radio technology, and the processing system may be programmed to determine distances between the emitting object and the detecting device by determining distances from received signal strength indicators of the detected signals.

The event may be one of a plurality of events, and wherein in attempting to associate each cluster with an event the processing system may be programmed to associate each cluster with any of the plurality of events.

In allocation of the cluster to one of the plurality of events the processing system may be programmed to allocate the cluster to the event closest to a mean position of the objects in the cluster. Alternatively, the processing system may be programmed to allocate the cluster to the event closest to a determined key object of the plurality of objects. In some cases, in allocation of the cluster to one of the plurality of events the processing system may be programmed to provide confidence scores for the allocation of the cluster to one of the plurality of the events and others of the plurality of events.

In determining clusters of the objects, the processing system may be programmed as follows: to determine whether two objects are within a predetermined proximity threshold, assign those two objects are a cluster, determine whether any further objects are within the predetermined proximity threshold of objects in the cluster and adding such further objects into the cluster, and perform the step of determining whether any further objects are within the predetermined proximity threshold of the objects in the cluster until an end condition is reached. One obvious end condition is lack of any further objects within the predetermined proximity threshold of any device in the cluster. However, another possible end condition is that a predetermined cumulative distance threshold defining maximum cluster size is reached.

### BRIEF DESCRIPTION OF FIGURES

Embodiments of the disclosure will now be described, by way of example, with reference to the following figures, in which:

FIG. 1 shows an exemplary environment in which embodiments of the disclosure may be employed;

FIG. 2 shows an electronic device adaptable for use in embodiments of the disclosure;

FIG. 3 shows in broad terms a method of associating objects using proximity sensing for use in the environment illustrated in FIG. 1;

FIG. 4 shows an initial scanning stage for detection of sensing devices in an exemplary implementation of the method of FIG. 3;

FIG. 5 shows data synchronisation in an exemplary implementation of the method of FIG. 3;

FIG. 6 illustrates schematically a clustering algorithm used in an exemplary implementation of the method of FIG. 3;

FIG. 7 illustrates a process for associating sensing devices with one event used in an exemplary implementation of the method of FIG. 3; and

FIG. 8 illustrates how the processes of FIGS. 4 to 7 can be used in a hospital context to allocate costs to patients.

### DETAILED DESCRIPTION

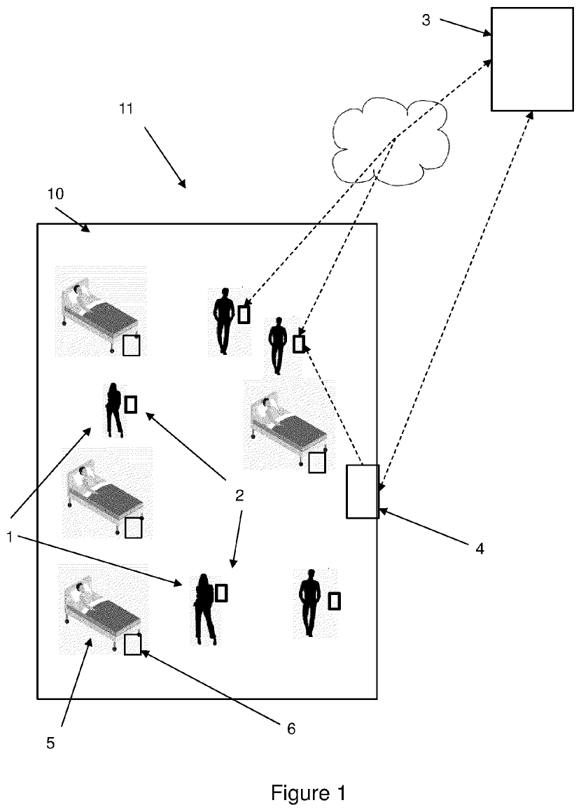

FIG. 1 illustrates an exemplary system 11 for object association using proximity sensing in an environment 10. In this case, the system is a hospital environment. Users 1 each bear an electronic device 2 adapted to emit signals using a suitable technology, such as short-range wireless. Hospital beds 5 with patients are here equipped with detectors 6—used in certain embodiments of the disclosure—adapted to receive signals emitted by user electronic devices. In certain embodiments of the disclosure, the electronic devices are adapted to receive signals emitted by other user electronic devices—in some embodiments, there may be both detectors 6 and electronic devices 2 adapted to do this.

These electronic devices 2 are here interacting over appropriate network connections with a cloud service 3 which can provide analysis and reporting relating to the assemblage of electronic devices 2. This is shown here as reporting back to a site server 4 which provides reporting for a site. Other computing architectures are possible, for example some or all of the computing carried out by the cloud service 3 could be carried out at the site server 4, using an edge computing approach.

In this case, the object association system 11 is one that could be used to identify interaction between medical professionals (with electronic devices 2) and patients in hospital beds 5. For example, each of the plurality of electronic devices 2 may include a wireless communication system for transmitting and receiving communication signals containing identifiers of the respective electronic device 2. The communication signals may, for example, take the form of advertisements and are referred to as such in the following description.

The electronic devices 2 may be devices that are specifically developed for advertising their presence, or they may be dual purpose devices with another function in the environment (for example, a user's security pass, which also serves to open doors within the environment). They may also be general-purpose computing or communication devices—such as a user's mobile telephone—running a suitable application, and accessing hardware already present in the general-purpose device. Furthermore, for convenient monitoring, each electronic device 2 may for example, take the form of, or be incorporated into, a wearable device.

When one of the electronic devices moves within range of a detecting device (which may in appropriate embodiments be either a detector 6 or an electronic device 2), a detecting device can detect the respective advertisements transmitted from an emitting electronic device and thereby it is possible to identify that electronic device based on the respective device identifier. As will be described further below, it is possible to measure the signal strength of the received advertisements and thereby to determine the proximity of the detected electronic device to the detecting device, for example based on a received signal strength indication (RSSI) of the advertisements.

In this manner, when two or more device-carrying individuals meet, the object association system 11 is able to determine the proximity of the emitting device to the detecting device, and the duration of this proximity, which can be recorded and used for various advantageous purposes. To minimise the risk of unobserved devices or contact events, each electronic device 2 may be configured to transmit the advertisements periodically, for example at a rate, or frequency, that is optimised for effective object association.

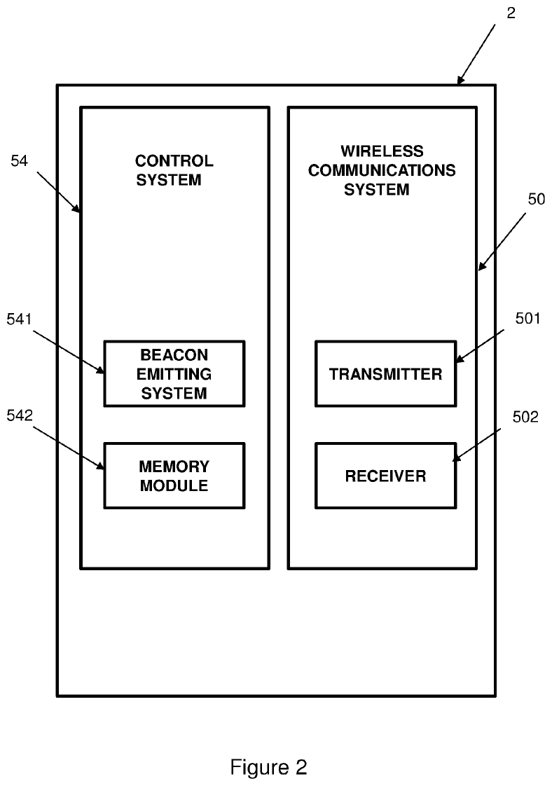

FIG. 2 illustrates a non-limiting example of such an electronic device 2. As shown in FIG. 2, the electronic device 2 here includes a wireless communication system 50 and a control system 54.

The wireless communication system 50 may be substantially as described above and is operable by the control system 54 to transmit advertisements for detection by the other electronic devices and to scan for counterpart advertisements transmitted from the other electronic devices. The advertisements may be transmitted on any suitable communication channel, including a Bluetooth® low energy, an Infrared, a WiFi, and/or an Ultrawide band, communication channel.

For this purpose, the wireless communication system 50 may include one or more transmitters 501 and one or more receivers 502, such as Bluetooth® Low Energy transmitters and receivers, as shown in FIG. 2.

The control system 54 is configured to control the wireless communication system 50 for object association. In particular, the control system 54 is configured to control the transmission of advertisements from the transmitter(s) 501 of the wireless communication system 50, and may in some embodiments also be configured to control scanning for advertisements, performed by the receiver(s) 502 of the wireless communication system 50.

The control system 54 here includes a memory storage module 542 comprising a contact database for storing records of detected advertisements, and/or electronic devices, including the device identifier, a signal strength and a timestamp associated with the detected contact. The memory storage module 542 may interact over appropriate network connections with the cloud service 3 for providing updates, corrections, or additions to the contact database. The wireless communications system 50 may also connect to the site server 4 to provide data consolidation of contact events.

For the purpose of receiving and/or storing such data, the memory storage module 542 may take the form of a computer-readable storage medium (e.g., a non-transitory computer-readable storage medium). The computer-readable storage medium may comprise any mechanism for storing information in a form readable by a machine or electronic processors/computational device, including, without limitation: a magnetic storage medium (e.g., floppy diskette); optical storage medium (e.g., CD-ROM); magneto optical storage medium; read only memory (ROM); random access memory (RAM); erasable programmable memory (e.g., EPROM and EEPROM); flash memory; or electrical or other types of medium for storing such information/instructions.

For purposes of this disclosure, it is to be understood that the functional systems, elements, and modules of the electronic device 2 described herein may each comprise a control unit or computational device having one or more electronic processors. A set of instructions could be provided which, when executed, cause said control unit(s) to implement the control techniques described herein (including the described method(s)). The set of instructions may be embedded in one or more electronic processors, or alternatively, the set of instructions could be provided as software to be executed by one or more electronic processor(s). The set of instructions may be embedded in a computer-readable storage medium (e.g., a non-transitory computer-readable storage medium) that may comprise any mechanism for storing information in a form readable by a machine or electronic processors/computational device, including, without limitation: a magnetic storage medium (e.g., floppy diskette); optical storage medium (e.g., CD-ROM); magneto optical storage medium; read only memory (ROM); random access memory (RAM); erasable programmable memory (e.g., EPROM and EEPROM); flash memory; or electrical or other types of medium for storing such information/instructions.

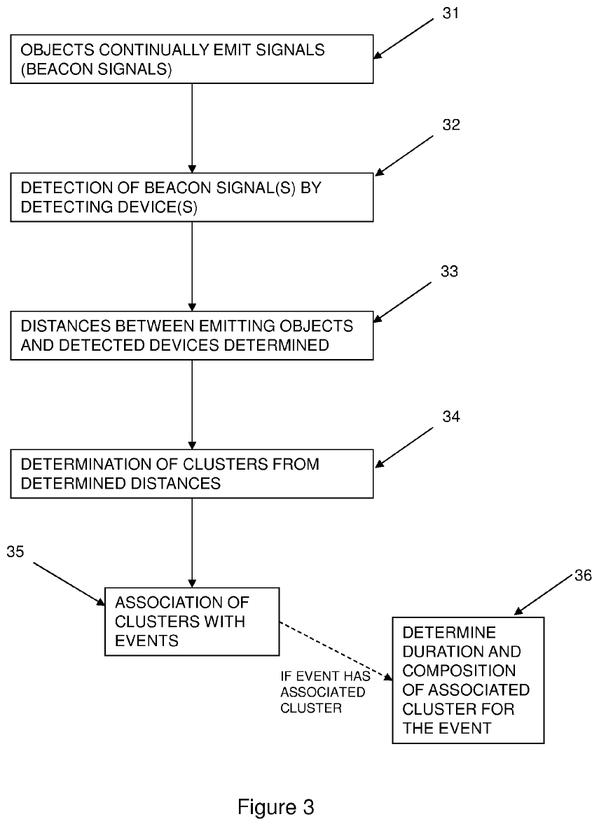

FIG. 3 shows in broad terms a method of associating objects using proximity sensing for use in the environment illustrated in FIG. 1. Each of the objects, where each object is in the FIG. 1 case an electronic device, continually emits 31 a signal comprising an identifier of that object—the objects thus act effectively as beacons. There are a number of ways in which this continual emission may be provided—for example, it may be periodic (e.g. every second, every 5 seconds) or the frequency of emission may vary depending on other factors, such as battery device level. This signal is emitted over a short-range communications technology, such as for example a short-range radio technology such as Bluetooth LE. A plurality of detecting devices detects 32 signals emitted by the objects. As noted above and discussed further below, these detecting devices may comprise the emitting electronic devices, other detectors disposed in the environment, or both. These detected signals are used to determine 33 distances between the emitting objects and the detecting devices. From an assemblage of these determined distances, clusters of the objects are determined 34. An attempt is then made for each cluster to associate 35 the cluster with an event in the environment-types of event occurring in the environment will be discussed further below. Where an association is made between a cluster and an event, the duration and composition of the cluster is determined 36.

The benefits of this approach can be seen in the context of a ward round in the hospital environment of FIG. 1. In a ward round, one or more medical professionals will travel around the hospital environment, spending time with individual patients. It may be desirable to determine whether particular medical professionals have seen particular patients, and when they did so, to ensure that medical records are complete. It may also be desirable to establish which medical professionals attended a particular patient, and for how long, to determine the amount of care that is in practice needed for particular conditions, or for accurate charging of medical professional time.

Conventionally, this process would need a high degree of intervention from medical professionals (signing of patient records and charts, for example), which is not an efficient use of time, and which could lead to errors. While automation by sensing would be desirable, this is potentially challenging, because the “most local” patient to a medical professional at any particular moment in time may not be the one that they are attending if the ward round involves a group of medical professionals of a significant size-some medical professionals may be closer to an adjoining patient as the senior consultant may be the only medical professional relatively sure to be close to the patient under consideration. In these circumstances, the time of the whole ward round team should typically be allocated to one patient at a time, in sequence during the course of the ward round.

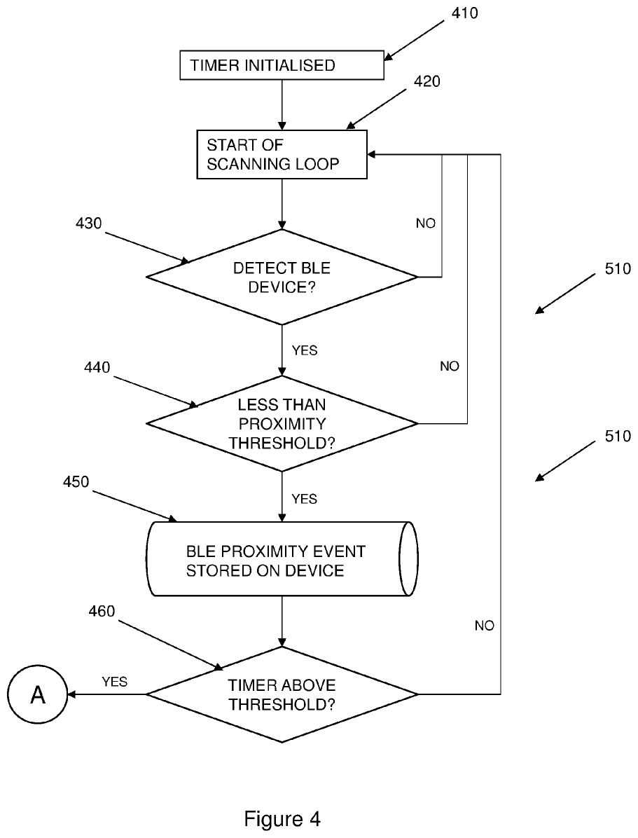

The approach described above achieves this objective, as it allows a cluster to be determined which will be the group of medical professionals involved in the ward round at any particular point in time, and it enables strategies which allow the time of the ward round group to be allocated to the correct patient. A suitable embodiment is described in detail with reference to FIGS. 4 to 7 below.

FIG. 4 shows an initial scanning stage for detection of sensing devices attached to users (in this case, medical professionals interacting with patients). This is carried out using an appropriate short-range communications technology—in the example described in detail here, this is Bluetooth Low Energy (BLE). Using this and similar technologies, distances between devices can be determined (generally, absolute position cannot be determined, though this may be a possibility with some technologies such as UWB). Other technologies—such as infrared or WiFi—could also in principle be used.

In the arrangement shown here, user devices both advertise and detect, so each device scans for other, nearby, devices. An alternative possibility is that user devices only advertise, but do not detect—detection may then be carried out by detectors in the environment, such as detectors shown as associated with patient beds in the FIG. 1 arrangement. A further possibility is for user devices to detect, but for there also to be detectors in the hospital environment.

Devices advertise by periodically emitting an advertising packet using the relevant communications technology. This advertising packet includes a unique device identifier—this may be associated with the user of the device in a separate database for later data reconciliation. The scanning process is shown in detail in FIG. 4. First of all, the time for the system of detecting devices is initialised 410, at which point a scanning loop is initiated 420. Once the scanning loop is initiated, the detective device detects signals 430 from another user device, here a BLE device, reverting to the beginning of the scanning loop if this is unsuccessful. If this step is successful and if such a device is detected, then a determination is made 440 whether the distance to the emitting device is less than a proximity threshold.

The strength of the signal may be expressed in terms of received signal strength index (RSSI). When the scanning process detects another device, an algorithm analyses the RSSI in order to determine whether the devices are within a predetermined proximity threshold of each other. A variety of strategies are available to do this, as the skilled person will appreciate. The algorithm may be a machine learning algorithm (such as a decision tree, random forest, or neural network). The algorithm may also comprise thresholding, averaging, or weighted averaging. The algorithm may comprise linear regression (to predict a continuous outcome such as distance in centimetres) or logistic regression (to estimate a binary outcome such as whether or not a physical distance exceeds a specified threshold).

If the result of the analysis is that the physical distance was not within the threshold, the scanning loop returns to the start as before. However, if the physical distance is found to lie within the threshold, this is identified as a proximity event and it is stored 450 along with a timestamp value. Associated data (for example, the device identifier, the RSSI and/or filtered RSSI, or simply a binary variable to indicate that devices are within a predetermined distance) can also be stored. Unless a timing or other threshold condition is met 460, the scanning loop again reverts to the start.

The threshold condition is used to initiate collection of data for further analysis. This may be set as a fixed period of time (say a 3-hour shift or a 12-hour working day), or it may be determined by quantity of stored data. In any event, this will be chosen to ensure that data is not lost, and that the objectives of the system will be met (in particular, that the data can be analysed, and results provided to meet any necessary timescale).

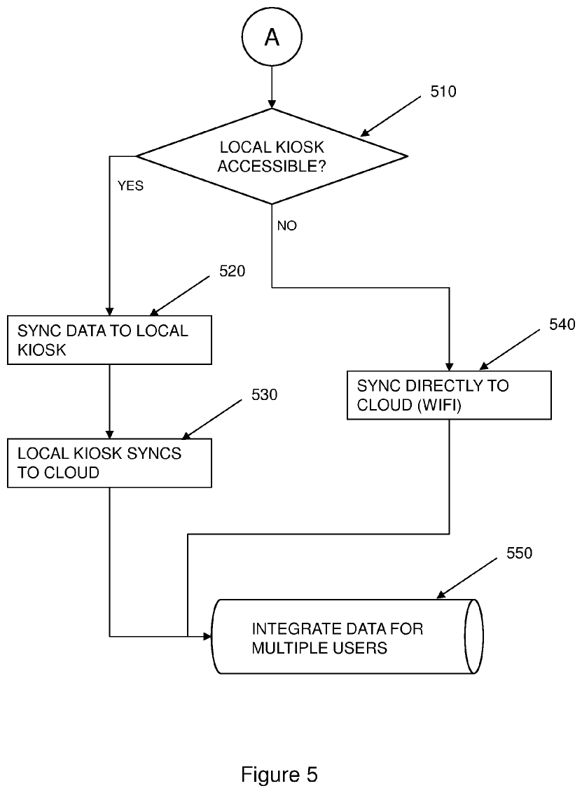

FIG. 5 shows a step of syncing data to the cloud. As noted above, this may occur after a certain volume of data is stored, or after a certain time duration has elapsed. First of all it may be determined 510 whether a local kiosk is available—this may be a tablet device or another form of computing device with internet connectivity. If so, data can be synced 520 to the local kiosk and subsequently synced 530 to the cloud from the local kiosk—if not, it may be synced directly 540 to the cloud, for example via WiFi. At the cloud, data from multiple sensor devices are aggregated 550 to form a dataset for multiple users.

While a cloud solution is described here, it should be noted that this could of course be done more locally, for example on a local server, or that a combination of edge and cloud servers could be used to provide a distributed solution.

The next step is the process of identifying clusters—in this case, clusters of clinicians (or other medical professionals). This and subsequent steps will typically occur at some time after the ward visit using data from the cloud or other aggregated data storage, preferably once all relevant data has been uploaded from individual user devices or other detectors.

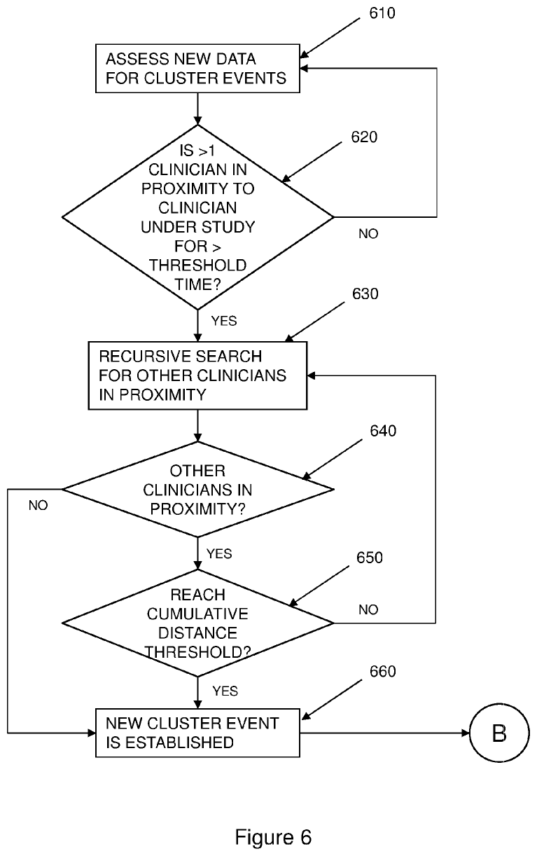

FIG. 6 illustrates schematically a clustering algorithm. The objective of the clustering algorithm is to identify the cluster of medical professionals interacting with a particular patient—the challenge is to ensure that when a larger number of clinicians are engaged with a patient, where a clinician may be standing quite far away at the back of the group, the clinician will be assigned to the same cluster and their time will be assigned to the correct patient. This is challenging because even though there might be little or zero wireless signal detected between patient and clinician, the clinician should still be assigned to that patient.

The algorithm can take a tree-like approach, in which a clinician is considered at the root of the process, and then each path to other clinicians is followed until the end of the clinician cluster is found (ie. until no signal is detected from any additional clinician device, or when the received signal strength (or filtered signal) from any such device falls below a certain threshold). The algorithm can also comprise a cumulative distance measure from the root that, if reached, means that path will be discarded (for example, the rule may be that in practice a clinician will not be more than 8 metres distant from the patient). This cumulative distance threshold may be calibrated for a particular environment based on the dimensions of hospital wards, or on the ward round practices, in the relevant hospital environment.

The clustering algorithm may be deployed when a clinician is found to be in proximity to two or more other clinicians. The algorithm can recursively search for additional clinicians until the entire cluster is identified, and no other clinicians are in proximity to any cluster members, or until the maximum distance threshold is reached.

As shown in FIG. 6, the first step is to look 610 for a new potential cluster event—this involves reviewing data until it is successfully determined 620 that for the clinician under scrutiny that there are more than one clinicians in proximity to that clinician for more than a threshold time. It should be noted that this is the trigger for determining whether there is a cluster, rather than for determining whether clinician time should be allocated to a patient—however, if no cluster is identified, clinician time may be allocated to a patient individually by clinician. If the conditions for a cluster are established in this step, then the size of the cluster is determined by recursively searching 630 for other clinicians in proximity until there are determined 640 to be no further clinicians in proximity or until it is determined 650 that a cumulative distance threshold is reached. If either of these ending conditions is reached, the cluster and its properties (composition, duration) is established 660.

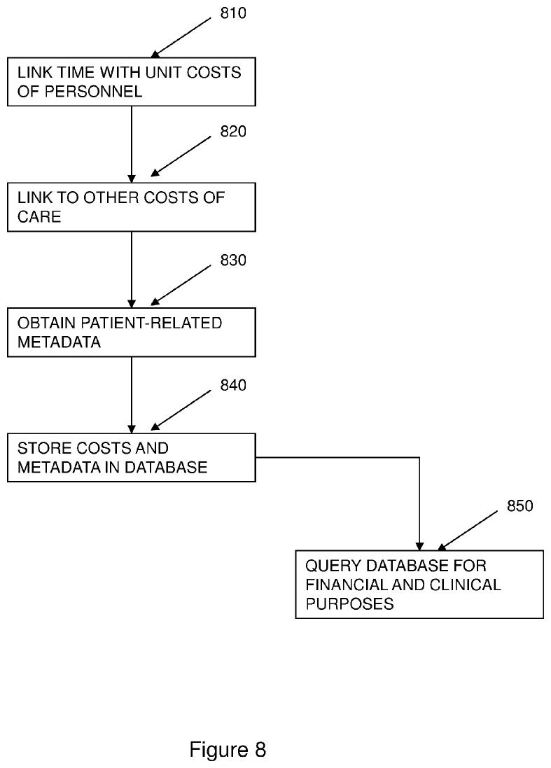

Once a cluster has been established, the entire cluster of clinicians will be allocated to one patient. In the context of FIG. 3, the patient is the “event”—the patient “event” is the presence of the patient in the hospital environment, typically in one bed in the environment for a particular period of time (the identity of the patient can typically be tracked through hospital records, so reconciliation of a cluster event with a specific patient can be done in later processing, as discussed below with reference to FIG. 8).

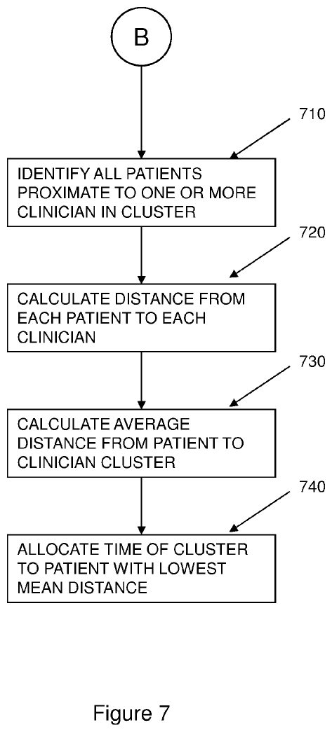

The process for allocating the cluster is shown in FIG. 7. There may be multiple patients in proximity with at least one member of the clinician cluster—the first step is to identify 710 all patients in proximity to one or more clinicians in the cluster. Analysis is needed to determine the most likely patient to whom cluster time should be allocated. This can be based on the distance metric (based on analysis of RSSI) between each patient and each member of the clinician cluster. The patient with the lowest mean distance to the clinician cluster members can be allocated the aggregate time of the clinician cluster. This is the approach taken in the FIG. 7 arrangement—the distance from each patient to each clinician is calculated 720, the aggregate distance from each patient to the clinician cluster is then calculated 730, and the cluster is allocated 740 to the patient with the lowest mean distance.

Other strategies to that shown in FIG. 7 may be employed. For example, there may be metadata in the database regarding relative seniority of the clinicians on a ward round. In this case, the patient who is nearest the senior physician leading the ward round may be assigned the time of the entire clinician cluster. This approach is based on the assumption that the senior physician (or other clinician) is the clinician most likely to be positioned close to the patient of interest. One possible approach is for the FIG. 7 approach to be followed initially, but for a correction to be made if suitable metadata exists and a senior physician can be identified.

Another approach can be taken if clinicians are categorised based on seniority, and the assumption is made that the most senior clinician is physically closer to the principal patient of interest than to any other patients. In this case, to associate a junior clinician to a patient we traverse the tree from that clinician to the clinician closer to her/him, and so on, until we reach a senior clinician. In this case, the first algorithmic objective is to reach the senior clinician, and not the patient. The disposition of the senior clinician relative to the patients is then used to identify the closest patient and to allocate the cluster.

A confidence score then can be calculated for each patient who is in proximity to at least one member of the clinician cluster. In cases where there is considerable uncertainty regarding the appropriate patient to whom to assign the cluster's time, the analysis can be supplemented with information from patient records and ward round notes. This data may be harvested from electronic health records in an automated manner, thereby improving the accuracy beyond that attainable by any single data source. Health records are often incomplete and lack data on the time, duration, and frequency of clinician-patient contact, yet often they will be sufficiently complete to indicate which clinicians are associated with that patient during a period of treatment. If, for example, the health records indicate that a patient is followed by clinicians A, B and C, but not clinician D, this information can be used in connection with the estimate produced by the wireless system (for example, to refine it, or to evaluate its quality).

If there are detecting devices associated with each patient bed, then the signals that they provide will be sufficient to allow these allocation strategies to work. If this is not the case—for example, if the only detecting devices are those carried by clinicians—then some mechanism may be needed to locate the clinicians accurately in the hospital environment. If short-range radio is used, for example, this may be used by a triangulation approach using known beacons in the environment, or it may be performed by a combination of technologies (for example, by combination of WiFi and GPS). The location of clinicians may then be compared to the known location of patient beds. Approaches to provide accurate location of a wireless device in a built environment are well known to the skilled person and will not be discussed further here—if the detecting device used by the clinician is a mobile telephone, an accurate geolocation method will typically already be built into the device.

FIG. 8 further describes how the resulting data can be used to allocate costs to patients. For each patient, the number of seconds of contact with each clinician is linked 810 to the appropriate unit cost of care associated with that clinician category. The cost per unit of time (e.g. dollars per second) may for example be calculated based on the annual salary of a staff grade band, and the number of hours of work scheduled per year. This cost of care from a salary perspective can be linked 820 to other costs of care such as medication, equipment, and overheads such as utilities and administration.

To provide full context for this information and to allow it to be used most effectively, it is also desirable to obtain 830 patient-level information such as diagnoses (e.g. using ICD-10 classification or diagnostic related groups), age, sex, and socioeconomic status. The results are stored 840 in a database as an estimated cost of care per day for each patient. The database would then be stored securely in accordance with local laws and regulations, e.g. HIPAA in the USA. This database can be queried 850 to ascertain the cost per day (or cost per hospital stay) of patients with a particular diagnosis or co-morbidities, or the cost segmented according to factors such as age. The database can also be queried to compare the amount of time spent with clinicians, and the cost of care, for each day of a patient's hospital stay—for example, there may be a higher cost per day for patients at the outset of hospital care and this may diminish during a post-operative phase, or alternatively patients who experience nosocomial infections or harmful errors in their care may experience a relatively high cost per day at a later stage of their hospitalization.

Information of this kind would be very difficult to establish without an information-gathering resource of this kind. Consequently, the hospital can use this patient-level query system to achieve new functionality, such as to model the impact on profitability by reducing the rate of medical errors, by improving efficiency of discharge, or by increasing the volume of certain categories of patients admitted to the hospital. It should of course be noted that potential benefits are not limited to financial benefits—the characteristics and durations of clinicians attending could also be stored or derivable, and this information could be used for clinical purposes. Information of this kind relating to the details of patient clinician use over time may for example have direct clinical benefits, such as refining treatment plans (for example, if it is found that including clinicians with particular specialties in a clinician group used for a particular patient type increases the effectiveness of treatment for that patient type).

The skilled person will appreciate that many further embodiments are possible within the spirit and scope of the disclosure set out here.

1. A method of associating objects using proximity sensing, where each of the objects to be associated is adapted to emit signals using a short-range communications technology, the method comprising:
each of the objects continually emitting a signal comprising an identifier of that object;
a plurality of detecting devices detecting signals emitted from the objects;
from detected signals from one or more of the detecting devices, determining distances between the emitting object and the detecting device for each of the detected signals;
from the determined distances, determining clusters of the objects;
attempting to associate each cluster with an event; and
for each cluster associated with the event, determining the duration and composition of the cluster.

2. The method of claim 1, wherein the plurality of detecting devices comprises at least one among:
one or more of the objects, and
a detecting device associated with a location of the event.

3. (canceled)

4. The method of claim 1, wherein the event is one of a plurality of events, and wherein attempting to associate each cluster with an event comprises attempting to associate each cluster with any of the plurality of events.

5. The method of claim 1, wherein the short-range communications technology is a radio technology.

6. The method of claim 5, wherein determining distances between the emitting object and the detecting device comprises determining distances from received signal strength indicators of the detected signals.

7. The method of claim 5, wherein the radio technology is Bluetooth Low Energy.

8. The method of claim 1, wherein determining clusters of the objects comprises determination of whether two objects are within a predetermined proximity threshold, assigning those two objects are a cluster, determining whether any further objects are within the predetermined proximity threshold of objects in the cluster and adding such further objects into the cluster, and performing the step of determining whether any further objects are within the predetermined proximity threshold of the objects in the cluster until an end condition is reached.

9. The method of claim 8, wherein one end condition is that a predetermined cumulative distance threshold defining maximum cluster size is reached.

10. The method of claim 4, wherein allocation of the cluster to one of the plurality of events comprises one among:
allocating the cluster to the event closest to a mean position of the objects in the cluster;
allocating the cluster to the event closest to a determined key object of the plurality of objects;
providing confidence scores for the allocation of the cluster to one of the plurality of the events and others of the plurality of events.

11-12. (canceled)

13. The method of claim 1, wherein the method is carried out in respect of a medical care environment, the objects are physically associated with medical professionals, and the events are each associated with a patient in the medical care environment.

14. A computing device having a processor, a memory, and a short-range communication apparatus, wherein the processor of the computing device is programmed to: continually emit a signal comprising an identifier for the computing device using the short-range communication apparatus; detect signals comprising identifiers of other computing devices using the short-range communication apparatus; and store in the memory a record of said detected signals for determination of whether the computing device and other computing devices have formed a cluster.

15. The computing device of claim 14, wherein the computing device is further adapted to upload the record of detected signals to a cluster determining resource when an upload condition is met.

16. The computing device of claim 15, wherein the upload condition is one of a data volume condition or a time condition.

17. The computing device of claim 14, wherein the short-range communication apparatus is a radio apparatus.

18. (canceled)

19. A computer system comprising a processing system, a memory and a communications system, wherein the processing system is programmed to: receive detection records from a plurality of detecting devices, wherein each detection record comprises time and signal information for detected signals comprising object identifiers of emitting objects, wherein each detection record is also associated with an object or location identifier of a detecting device; determine from the detection records distances between the emitting object and the detecting device for each of the detected signals; from the determined distances, determine clusters of the objects; attempt to associate each cluster with an event; and for each cluster associated with the event, determine the duration and composition of the cluster.

20. The computer system of claim 19, wherein, wherein the short-range communications technology is a radio technology, and wherein the processing system is programmed to determine distances between the emitting object and the detecting device by determining distances from received signal strength indicators of the detected signals.

21. The computer system of claim 19, wherein the event is one of a plurality of events, and wherein in attempting to associate each cluster with an event the processing system is programmed to associate each cluster with any of the plurality of events.

22. The computer system of claim 21, wherein for allocation of the cluster to one of the plurality of events, the processing system is programmed for one among:
allocating the cluster to the event closest to a mean position of the objects in the cluster;
allocating the cluster to the event closest to a determined key object of the plurality of objects;
providing confidence scores for the allocation of the cluster to one of the plurality of the events and others of the plurality of events.

23-24. (canceled)

25. The computer system of claim 19, wherein in determining clusters of the objects the processing system is programmed to determine whether two objects are within a predetermined proximity threshold, assign those two objects to a cluster, determine whether any further objects are within the predetermined proximity threshold of objects in the cluster and adding such further objects into the cluster, and perform the step of determining whether any further objects are within the predetermined proximity threshold of the objects in the cluster until an end condition is reached.

26. The computer system of claim 25, wherein one end condition is that a predetermined cumulative distance threshold defining maximum cluster size is reached.
